(19) United States
(12) Patent Application Publication
(10) Pub. No.: US 2017/0160290 A1
(43) Pub. Date: Jun. 8, 2017

(54) A NEW BIOMARKER OF CHRONIC ALLOGRAFT NEPHROPATHY AND OF RENAL TRANSPLANT REJECTION

(71) Applicants: INSERM (INSTITUT NATIONAL DE LA SANTÉ ET DE LA RECHERCHE MÉDICALE, Paris (FR); UNIVERSITÉ PIERRE ET MARIE CURIE (PARIS 6), Paris (FR); ASSISTANCE PUBLIQUE-HÔPITAUX DE PARIS (APHP), Paris (FR); FONDAZIONE IRCCS CA' GRANDA OSPEDALE MAGGIORE POLICLINICO, Milano (IT)

(72) Inventors: Christos CHATZIANTONIOU, Paris (FR); Jean-Claude DUSSAULE, Paris (FR); Piergiorgio MESSA, Paris (FR); Carlo ALFIERI, Paris (FR)

(21) Appl. No.: 15/323,997

(22) PCT Filed: Jul. 8, 2015
(86) PCT No.: PCT/EP2015/065565

§ 371 Date: Jan. 5, 2017

(30) Foreign Application Priority Data
Jul. 9, 2014 (EP) 14306120.8

(51) Int. Cl. G01N 33/68

(52) U.S. Cl. CPC G01N 33/6893; G01N 2800/245; G01N 2800/50; G01N 2333/70589; G01N 2333/705

(57) ABSTRACT

The invention relates to a method for determining whether a renal transplanted patient is at risk of transplant rejection or CAN, comprising a step of determining the expression level of the periostin (POSTN) gene on a renal transplant biopsy obtained from said transplanted patient. The invention also relates to a compound selected from the group consisting of monoclonal anti-CD20 antibodies, anti-thymocyte globulin, proteasome inhibitors, anti-C antibodies, monoclonal anti-CD3 antibodies, glucocorticoids, cytostatics, calcineurin inhibitors and mTOR inhibitors, for use in a method for preventing renal transplant rejection and in CAN development in a transplanted patient determined as being at risk of renal transplant rejection according to a method of the invention.

### FIELD OF THE INVENTION

The invention relates to the prediction or diagnostic of chronic allograft nephropathy (CAN) and/or of renal transplant rejection. Once the CAN and/or the rejection are diagnosed and/or predicted, methods for preventing CAN and/or the rejection are also provided.

### BACKGROUND OF THE INVENTION

Kidney transplantation (KTx) is the best therapy in presence of chronic kidney disease. Renal biopsy gives several clinical information about the graft and its prognosis. Renal transplant recipients may suffer from degenerative lesions denominated Chronic Allograft Nephropathy (CAN) and ultimately from graft rejections, such as T cell-mediated rejection (TCMR) and antibody-mediated rejection (ABMR), Renal graft rejections, including TCMR and ABMR, are usually diagnosed on the basis of histologic evaluation performed in response to biochemical evidence of graft impairment (e.g., elevated creatinine levels).

Histopathological evaluation of biopsy tissue is also the gold standard for the diagnosis of CAN, while prediction of the onset of CAN is currently impossible. CAN diagnosis is often based on observer-dependent interpretation of unspecific histological alterations, and patient prognosis remains ill-defined. The diagnosis of, for example, renal allograft rejection is made usually by the development of graft dysfunction (e.g., an increase in the concentration of serum creatinine) and morphologic evidence of graft injury in areas of the graft also manifesting mononuclear cell infiltration. Accurately predicting and diagnosing the outcome of CAN episode is crucial in the trend to optimize treatments and prevent the development of CAN and kidney loss of function.

There is a need to develop more accurate and noninvasive tests for predicting renal graft outcome including CAN that may allow earlier detection and intervention in transplanted patients.

### SUMMARY OF THE INVENTION

In a first aspect, the invention relates to a method for predicting or diagnosing chronic allograft nephropathy (CAN) in a renal transplanted patient, comprising a step of determining the expression level of the POSTN gene on a renal graft biopsy obtained from said transplanted patient.

In a second aspect, the invention relates to a method for determining whether a renal transplanted patient is at risk of transplant rejection, comprising a step of determining the expression level of the POSTN gene on a renal transplant biopsy obtained from said transplanted patient.

In a third aspect, the invention relates to the use of POSTN polypeptide as a biomarker of CAN and/or renal transplant rejection.

In a fourth aspect, the invention relates to a kit comprising means for determining the expression level of POSTN gene and means for determining the expression level of CD45 gene and/or Vimentin gene.

In a fifth aspect, the invention relates to a method for adjusting the immunosuppressive treatment administered to a renal transplanted recipient following its transplantation, comprising the following steps of: (i) performing the method for determining whether a renal transplanted patient has or is at risk of CAN and/or is at risk of transplant rejection of the invention, and (ii) adjusting the immunosuppressive treatment.

In a last aspect, the invention relates to a compound selected from the group consisting of monoclonal anti-CD20 antibodies, anti-thymocyte globulin (ATG), proteasome inhibitors, anti-C5 antibodies, monoclonal anti-CD3 antibodies, glucocorticoids, cytostatics, calcineurin inhibitors (CNI) and mTOR inhibitors, for use in a method for preventing CAN and/or renal transplant rejection in a transplanted patient determined as having or being at risk of CAN and/or being at risk of renal transplant rejection according to the invention.

### DETAILED DESCRIPTION OF THE INVENTION

The inventors have demonstrated following a clinical study performed on one hundred and forty nine kidney biopsies (KBx) for clinical reasons in KTx patients (M 51%; mean age 44±14 yrs/time of KTx 6±10 yrs) between 2009 and 2012 that periostin (POSTN) is useful for predicting earlier graft outcome and earlier graft rejection. Clinical and biochemical data were collected at the moment (T0), 6 and 12 months before and after the KBx. A follow up time of 12 months was considered. During the follow-up time, 32 patients restarted dialysis (D+). D+ had lower eGFR at the time of biopsy, and significantly higher positivity for CD45, VIM and POST than the others patients. However, POSTN is not related with renal function at T0 and its prognostic role is independent from creatinine and estimated glomerular filtration rate (eGF) and is stronger marker (best marker in sensitivity and specificity in predicting D+) than the two others markers studied, namely CD45 and Vimentin (VIM), which are already known as powerful markers for predicting renal transplant damages (AUC: CD45=0,678; VIM=0,673; POSTN=0,760) and renal transplant rejection.

### DEFINITIONS

Throughout the specification, several terms are employed and are defined in the following paragraphs.

As used herein, the term “periostin gene” (POSTN) also known as “osteoblast specific factor 2” (OSF-2) refers to gene encoding a protein known as a ligand for alpha-V/beta-3 and alpha-V/beta-5 integrins to support adhesion and migration of epithelial cells. The term “periostin gene” includes naturally occurring periostin as well as variants thereof. For instance, the naturally occurring human POSTN isoform 1 of 836 amino acids has an amino acid sequence as shown in GenPept database under accession number NP_006466.2 Other human POSTN isoforms 2, 3 and 4 has an amino acid sequence as shown in GenPept database under accession number NP_001129406.1, NP_001129407.1 and NP_001129408.1.

As used herein, the term “gene” refers to a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, the term “determining” includes qualitative and/or quantitative determination (i.e. detecting and/or measuring the expression level) with or without reference to a control or a predetermined value. As used herein, “detecting” means determining if POSTN is present or not in a biological sample and “measuring” means determining the amount of POSTN in a biological sample. As used herein, the term “biological sample” has its general meaning in the art and refers to any biological sample which may be obtained from a subject for the purpose of in vitro evaluation. Typically the expression level may be determined for example by immunohistochemistry (IHC) performed on renal transplant biopsy obtained from said transplanted patient.

The term “transplantation” and variations thereof refers to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from a human of the same or different genetic origins. In another scenario, the graft is derived from a species different from that into which it is transplanted, including animals from phylogenically widely separated species.

As used herein, the terms “kidney graft”, “renal graft”, “kidney transplant” or “renal transplant” are used herein interchangeably and refer to the organ (i.e. the kidney) which is transplanted to a patient suffering from End-Stage Renal Disease (ESRD).

As used herein, the term “predetermined reference value” refers to the amount of POSTN in biological samples obtained from the general population or from a selected population of subjects. The predetermined reference value can be a threshold value or a range. For example, the selected population may be comprised of apparently healthy transplanted patient, such as individuals who have not previously had any sign or symptoms indicating the outcome of a kidney transplant rejection or chronic allograft nephropathy (CAN).

As used herein, the term “risk” refers to the probability that an event will occur over a specific time period, such as the onset of transplant rejection, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a patient compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk determination” in the context of the invention encompasses making a prediction of the probability, odds, or likelihood that an event may occur. Risk determination can also comprise prediction of future clinical parameters, traditional laboratory risk factor values, such age, sex mismatch, HLA-testing, etc. . . . ; either in absolute or relative terms in reference to a previously measured population. The methods of the invention may be used to make categorical measurements of the risk of transplant rejection, thus defining the risk spectrum of a category of transplanted patient defined as being at risk of transplant rejection.

### Prognostic Methods of the Invention

In a first aspect, the invention relates to a method for predicting or diagnosing chronic allograft nephropathy (CAN) in a renal transplanted patient, comprising a step of determining the expression level of the POSTN gene on a renal graft biopsy obtained from said transplanted patient.

As used herein, the term “chronic allograft nephropathy” (CAN) refers to the leading cause of late graft loss (around 3-5% per year). CAN manifests itself as a slowly progressive decline in glomerular filtration rate, usually in conjunction with proteinuria and arterial hypertension. This disorder represents a consequence of combined immunological injury (e.g., chronic rejection) and non-immunological damage (e.g., hypertensive nephrosclerosis, or nephrotoxicity of immunosuppressive drugs like CsA), ultimately leading to fibrosis and sclerosis of the graft, associated with progressive loss of kidney function.

In one embodiment, said method comprises a step of (i) determining the expression level of the periostin (POSTN) gene in a renal graft biopsy obtained from said transplanted patient, and (ii) comparing said expression level with a predetermined reference value, wherein an increase in the expression level of the POSTN gene is indicative of having or being at risk of chronic allograft nephropathy (CAN).

In a second aspect, the invention relates to a method for determining whether a renal transplanted patient is at risk of transplant rejection, comprising a step of determining the expression level of the POSTN gene on a renal transplant biopsy obtained from said transplanted patient.

As used herein, the terms “transplant rejection” or “graft rejection” encompass both acute and chronic transplant rejection. “Acute transplant rejection” is the rejection by the immune system of a tissue transplant recipient when the transplanted tissue is immunologically foreign. Acute transplant rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute transplant rejection can be inhibited or suppressed with immunosuppressive drugs such as rapamycin, cyclosporin and the like. “Chronic transplant rejection” generally occurs in humans within several months to years after engraftment, even in the presence of successful immunosuppression of acute rejection. Fibrosis is a common factor in chronic rejection of all types of organ transplants.

In one embodiment, the transplant rejection is an acute transplant rejection or a chronic transplant rejection

In one embodiment, the transplant rejection is an antibody-mediated rejection (ABMR) or a T cell-mediated rejection (TCMR).

In a particular embodiment, the transplant rejection is an acute ABMR.

In another particular embodiment, the transplant rejection is a chronic ABMR.

In a particular embodiment, the transplant rejection is an acute TCMR.

In another particular embodiment, the transplant rejection is a chronic TCMR.

In one embodiment, said method comprises a step of (i) determining the expression level of the periostin (POSTN) gene in a renal transplant biopsy obtained from said transplanted patient, and (ii) comparing said expression level with a predetermined reference value, wherein an increase in the expression level of the POSTN gene is indicative of having or being at risk of transplant rejection.

In another aspect, the invention relates to a method for predicting or determining the renal graft status or outcome, comprising a step of determining the expression level of the periostin (POSTN) gene on a renal graft biopsy.

The term “predicting or determining kidney graft status or outcome” refers to a process aimed at one or more of: determining if the kidney graft of a patient is stable or not (i.e. with graft dysfunction and/or graft lesions), and/or predicting the survival or the failure of the kidney graft. Accordingly, the graft status or outcome may comprise rejection, tolerance, non-rejection based allograft injury, graft function, graft survival or chronic graft injury.

In one embodiment, said method comprises a step of (i) determining the expression level of the periostin (POSTN) gene in a renal graft biopsy obtained from said transplanted patient, and (ii) comparing said expression level with a predetermined reference value, wherein an increase in the expression level of the POSTN gene is indicative of that kidney graft is not stable (i.e. with graft dysfunction and/or graft lesions).

### Methods for Determining the Expression Level of the Biomarker of the Invention

Determination of the expression level of periostin (POSTN) gene may be performed by a variety of techniques. Generally, the expression level as determined is a relative expression level. For example, the determination comprises contacting the biological sample with selective reagents such as probes, primers or ligands, and thereby detecting the presence, or measuring the amount, of polypeptide or nucleic acids of interest originally in said biological sample. Contacting may be performed in any suitable device, such as a plate, microtiter dish, test tube, well, glass, column, and so forth. In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the biological sample.

In a particular embodiment, the expression level of the POSTN gene may be determined by determining of the quantity of mRNA.

For example the nucleic acid contained in the renal transplant biopsy is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical.

In certain embodiments, it will be advantageous to use nucleic acids in combination with appropriate means, such as a detectable label, for detecting hybridization.

A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

In a particular embodiment, the methods of the invention comprise the steps of providing total RNAs extracted from a biological sample such a renal transplant biopsy and subjecting the RNAs to amplification and hybridization to specific probes, more particularly by means of a quantitative or semi-quantitative RT-PCR.

In a particular embodiment, the expression level of the POSTN gene may be determined by determining of the quantity of proteins encoded by the POSTN gene.

Such methods comprise contacting the biological sample with a binding partner capable of selectively interacting with the protein present in said sample. The binding partner is generally an antibody that may be polyclonal or monoclonal, preferably monoclonal. Polyclonal antibodies directed against POSTN are well known from the skilled man in the art such as the antibodies commercialized by Abcam ab14041, Santa-Cruz sc-67233 and Biovendor R&D RD184045100. Monoclonal antibodies directed against POSTN are also well known such as the monoclonal antibodies commercialized by Sigma SAB4200197 or described in the international patent applications WO 03/016471 and WO 2012/083132.

As used herein, the term “monoclonal antibody” refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the invention.

Laboratory methods for preparing monoclonal antibodies are well known in the art (see, for example, Harlow et al., 1988). Monoclonal antibodies (mAbs) may be prepared by immunizing purified POSTN into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993).

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

As used herein, the term “labelled” with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or indocyanine (Cy5), to the antibody or aptamer, as well as indirect labelling of the probe or antibody (e.g., horseradish peroxidise, HRP) by reactivity with a detectable substance. An antibody or aptamer may be also labelled with a radioactive molecule by any method known in the art. For example, radioactive molecules include but are not limited radioactive atom for scintigraphic studies such as I¹²³, I¹²⁴, In¹¹¹, Re¹⁸⁶ and Re¹⁸⁸.

More particularly, an immunohistochemistry (IHC) method may be used.

IHC specifically provides a method of detecting a target protein in a biological sample or tissue specimen in situ. The overall cellular integrity of the sample is maintained in IHC, thus allowing detection of both the presence and location of the protein of interest. Typically a biological sample is fixed with formalin, embedded in paraffin and cut into sections for staining and subsequent inspection by light microscopy. Current methods of IHC use either direct labelling or secondary antibody-based or hapten-based labelling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

In a further embodiment of the invention, methods of the invention comprise measuring the expression level of at least one further gene of interest.

For example, the other gene of interest may be CD45 gene and Vimentin gene.

Yet another aspect of the invention relates to a kit for performing a method of the invention, said kit comprising means for determining the expression level of POSTN gene in a renal transplanted biopsy obtained from a patient. The kit may include an antibody or an aptamer as above described. In a particular embodiment, the antibody or aptamer is labelled as above described. The kit may also contain other suitably packaged reagents and materials needed for the particular detection protocol, including solid-phase matrices, if applicable, and standards. The kit may also contain one or more means for determining the expression level of at least one further gene if interest such as CD45 and/or Vimentin (such as anti-CD45 and/or Anti-Vimentin antibody or aptamer, labelled or not).

The invention also relates to the use of a kit of the invention for determining whether a renal transplanted patient has or is at risk of CAN and/or is at risk of transplant rejection.

The invention relates to the use of a kit comprising means for determining the expression level of POSTN gene (such as an labelled anti-POSTN antibody) in a renal transplant biopsy obtained from a renal transplanted patient for performing a method for determining whether a renal transplanted patient is at risk of transplant rejection.

In still further embodiment of the invention, methods of the invention comprise measuring at least one further physiological parameter.

The term “physiological parameter”, as used herein, refers generally to any parameter that may be monitored to determine one or more quantitative physiological levels and/or activities associated with the patient.

For example, such physiological parameter may be selected from the group consisting of estimated glomerular filtration rate (eGFR) value, mineral metabolism related parameters (such as Vitamin D), and the like.

In another aspect, the invention relates to the use of POSTN polypeptide as a biomarker of chronic allograft nephropathy (CAN) and/or renal transplant rejection.

The term “biomarker”, as used herein, refers generally to a molecule, i.e., a gene (or nucleic acid encoding said gene), protein, the expression of which in a biological sample from a patient can be detected by standard methods in the art (as well as those disclosed herein), and is predictive or denotes a condition of the patient from which it was obtained.

### Methods for Adjusting an Immunosuppressive Treatment

The invention further provides methods for developing personalized treatment plans. Information gained by way of the methods described above can be used to develop a personalized treatment plan for a renal transplant recipient.

Accordingly, in a further aspect, the invention relates to a method for adjusting the immunosuppressive treatment administered to a renal transplanted recipient following its transplantation, comprising the following steps of: (i) performing the method for determining whether a renal transplanted patient has or is at risk of chronic allograft nephropathy (CAN) and/or is at risk of graft rejection of the invention, and (ii) adjusting the immunosuppressive treatment.

The methods can be carried out by, for example, using any of the methods for determining risk described above and, in consideration of the results obtained, designing a treatment plan for the transplant recipient. If POSTN is present in the renal transplant biopsy obtained from a patient of interest, this indicates that said patient is at risk for an undesirable clinical outcome (e.g., CAN and/or renal transplant rejection). Therefore, said patient is a candidate for treatment with an effective amount of an immunosuppressive treatment (e.g. by an anti-rejection agent). On the contrary, the absence of POSTN in the renal transplant biopsy is indicative of a reduced risk of CAN and/or transplant rejection. Moreover, depending on the expression level of POSTN (i.e. low level or high level of POSTN in the analyzed biological sample), the patient may require a treatment regime that is more or less aggressive than a standard regimen, or it may be determined that the patient is best suited for a standard regimen. For instance, a patient with a low level of POSTN may avoid an immunosuppressive treatment (or require a less aggressive regimen) and their associated side effects.

In one embodiment, such reduction and protection comprise a therapeutic intervention with the patient such as administration of anti-thymocyte globulin (ATG), monoclonal anti-CD20 antibodies (rituximab), proteasome inhibitor (bortezomib), anti-C5 antibodies (eculizumab), intravenous administration of immunoglobulins, plasmapheresis, monoclonal anti-CD3 antibodies (muromonab), glucocorticoids, cytostatics (mycophenolate mofetil (MMF), mycophenolic acid (MPA), azathioprine (AZA)), calcineurin inhibitors (CNI) (ciclosporin) and mTOR inhibitors (sirolimus and tacrolimus).

### Therapeutic Methods and Uses

In a further aspect, the invention relates to a method for preventing chronic allograft nephropathy (CAN) and/or renal transplant rejection-in a transplanted patient, comprising the following steps of: (i) performing the method for determining whether a renal transplanted patient is at risk of renal transplant rejection of the invention, and (ii) administering to said patient an immunosuppressive treatment.

In one embodiment, the immunosuppressive treatment administered to the patient is a therapeutically effective amount of at least one compound selected from the group consisting of anti-thymocyte globulin (ATG), monoclonal anti-CD20 antibodies (rituximab), proteasome inhibitors (bortezomib), anti-C5 antibodies (eculizumab), monoclonal anti-CD3 antibodies (muromonab), glucocorticoids, cytostatics (mycophenolate mofetil (MMF), mycophenolic acid (MPA), azathioprine (AZA)), calcineurin inhibitors (CNI) (cyclosporine, tacrolimus) and mTOR inhibitors (sirolimus, everolimus, rapamycin).

In one particular embodiment, the immunosuppressive treatment is composed by an inductive therapy comprising a therapeutically effective amount of anti-thymocyte globulin (ATG), monoclonal anti-CD20 antibodies (rituximab) and/or proteasome inhibitors (bortezomib); and a maintenance therapy comprising a therapeutically effective amount of glucocorticoids, cytostatics (mycophenolate mofetil (MMF), mycophenolic acid (MPA), azathioprine (AZA)), calcineurin inhibitors (CNI) (cyclosporin, tacrolimus) and mammalian target of rapamycin inhibitors (mTOR) (sirolimus, everolimus, rapamycin), and/or anti-05 antibodies (eculizumab).

By “therapeutically effective amount” is meant an amount sufficient to achieve a concentration of compound which is capable of preventing or slowing down the disease to be treated. Such concentrations can be routinely determined by those of skilled in the art. The amount of the polypeptide actually administered will typically be determined by a physician or a veterinarian, in the light of the relevant circumstances, including the condition to be treated, the chosen route of administration, the actual compound administered, the age, weight, and response of the patient, the severity of the subject's symptoms, and the like. It will also be appreciated by those of skilled in the art that the dosage may be dependent on the stability of the administered compound.

The compounds of the invention may be administered by any means that achieve the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, buccal, topical, local, inhalant or subcutaneous use. Parenteral and topical routes are particularly preferred.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

The doses used for the administration can be adapted as a function of various parameters, and in particular as a function of the mode of administration used, of the relevant pathology, or alternatively of the desired duration of treatment. For example, it is well within the skill of the art to start doses of the compounds at levels lower than those required to achieve the desired therapeutic effect and to gradually increase the dosage until the desired effect is achieved. However, the daily dosage of the polypeptides may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day.

The invention will be further illustrated by the following figures and examples. However, these examples and figures should not be interpreted in any way as limiting the scope of the present invention.

### FIGURES

FIG. 1: ROC curve CD45 (A), Vimentin (B) and Periostin (C) as predictors of restart dialysis in the year after KBx.

### EXAMPLE

Material & Methods

Study Cohort Characteristics:

The study was performed in 149 (Kidney Transplanted) KTx patients, who were submitted to a renal biopsy (Bx) in our Department during the period from February 2009 to August 2012 and followed-up until 30^{th} of August 2013 or up to the return on dialysis or death. The protocol was approved by the Ethic Committee of Fondazione IRCCS Policlinico and was conducted according to the ethical principles of the Helsinki Convention.

Study Design:

Indication to the Renal Graft Biopsy:

The Bx were performed according to the approved local procedure, with a 16 Gauge needle and under ultrasound control. The Bx were performed only on clinical indications, which were as follows: isolated proteinuria (15%); isolated reduced renal function (RF), assessed by increased serum creatinine >25% respect the basal level of each patient (60%); association of both proteinuria and reduced RF (14%); other clinical reasons (suspected BK nephropathy, delayed graft function after KTx, protocol Bx) in the remaining 11%.

Evaluated Clinical and Laboratory Parameters:

Medical history and basal clinical evaluation were recorded at time of Bx. The clinical assessment in each patient was repeated according to our in-centre protocol at each ambulatory visit. At each control visit the following parameters were evaluated:

Clinical Parameters:

Body weight, systolic blood pressure (SBP), diastolic blood pressure (DBP)

Routine Laboratory Evaluation:

Uric acid, haemoglobin, albumin, glycaemia, cholesterol, triglycerides, CRP, serum Creatinine, Proteinuria/24 h (UProt/24 h), eGFR

Mineral Metabolism Related Parameters:

Calcium, Phosphate, PTH, Alkaline Phosphatase (ALP)

PTH and 25(OH)D levels were assessed at the time of Bx. The 25(OH)D values were available in only 118 patients due to technical reasons.

Analytical Procedures:

Biological samples were obtained in our Department from each patient after 12 hour fasting condition, and all biochemical analyses were performed in the same laboratory at our Institution.

Serum creatinine was determined by Jaffè method and UProt/24 h by immunoturbidimetric method. Estimated glomerular filtration was determined using the Modification of Diet in Renal Disease (MDRD) formula (1). DSA were assessed by routine methodology (2, 3).

PTH

For the dosage of intact PTH we used ECLIA (ImmunoAssay in ElettroChemiLuminescent) method by Roche by means of modular analytics E170. The Measure range was 1.20-5000 pg/mL The Conversion was: pg/mL×0.106=pmol/L. Normal range: 15-65 pg/ml.

Vitamin D

Levels of 25(OH)D have been assessed in serum specimens by enzyme-immunoassay (Kit EIA AC-57FI-immunodiagnostic system Boldon, UK), using a highly specific sheep 25(OH)D antibody and enzyme (horseradish peroxidase) labelled avidin. The sensitivity threshold was 5 nmol/ml (2 ng/ml). The specificity of the antiserum was assessed with the following analytes at 50% binding of the zero calibrator; cross reactivity: 25-Hydroxyvitamin D3 100%; 25-Hydroxyvitamin D2 75%; 24, 1,25-Dihydroxyvitamin D3 100%; Cholecalciferol (D3)<0.01%; Ergocalciferol (D2)<0.30%. The intra-assay precision was calculated from 10 duplicate determinations of two samples each, performed in a single assay (CV between 5.3% and 6.7%). The inter-assay precision was calculated from duplicate determinations of two samples performed in 11 assays (CV between 4.6% and 8.7%). This method has been demonstrated to give results strictly related to the ones obtained by two different RIA methods, which are considered gold-standard for 25-OH-vitamin D assessment (EIA method vs method 1RIA and vs method 2 RIA (http://www.idsplc.com/int/products/25-hydroxy-vitamin-d-eia-ac-57fl#description).

All the other biochemical parameters were evaluated according to routine methodology used in our central laboratory.

Histological Analyses:

Tissue samples, fixed in 4% buffered paraformaldehyde and embedded in paraffin, were processed following the International guidelines (4) and examined by light microscopy and immune-staining. Electronic microscopy was performed in selected cases on clinical indication. Routine light microscopy staining were conducted according to standard methods.

Histological diagnoses were based on Banff '97 (updated Banff '09) diagnostic criteria (5, 6). Histological diagnoses were grouped as Normal, Antibody-mediated Rejection (either acute or chronic), T-cell mediated Rejection (either acute or chronic), CAI (chronic allograft injury), Glomerulonephritis (“de novo” or relapsed), other diagnoses (not specific and/or significant lesions).

For statistical purposes, we evaluated the degree of glomerular sclerosis, tubular atrophy (TA), interstitial fibrosis (IF) and interstitial inflammation (I-Inf) according to the following criteria:

Glomerular sclerosis was evaluated as the percentage of sclerotic glomeruli in each sample. Conversely, TA, IF and interstitial inflammation were qualitatively graded as absent, mild, moderate and severe in each sample.

Specific Staining:

An avidin-biotin immunoperoxidase staining method was applied to detect CD45 and Vimentin on 5 μm thick tissue sections. Briefly, sections were rehydrated, then immersed in 10 mM citrate buffer (pH 6.0), treated with microwave irradiation at 500 W for 10 min, and cooled at RT. After incubation with 0.5% avidin (Sigma-Aldrich, Milan, Italy) and 0.01% biotin (Sigma-Aldrich), to suppress endogenous avidin-binding activity, a 3% H₂O₂ solution was applied to block endogenous peroxidase. After washing, sections were sequentially incubated with the primary antibody mouse anti-human CD45 (Dako, Milan, Italy), mouse anti-Vimentin (Invitrogen, Milan, Italy) and rabbit anti-human Periostin (Biovendor Brno Reckovice, Czech Republic), with the secondary biotinylated antibody (Invitrogen) and with the peroxidase-labeled streptavidin (Invitrogen). Peroxidase activity was detected with 3,5-diaminobenzidine (Sigma-Aldrich), and sections were counterstained with Harry's hematoxylin, dehydrated and mounted in Entellan (Bio-Optica, Milan, Italy). Specificity of antibody labelling was demonstrated by the lack of staining after substituting phosphate buffer saline (PBS) and proper control immunoglobulins (Invitrogen) for the primary antibody.

Quantitative Evaluation:

Images were acquired by a Zeiss Axioscope 40FL microscope, equipped with AxioCam MRc5 digital videocamera and immunofluorescence apparatus (Carl Zeiss SpA). Quantitative evaluation was performed using the AxioVision analysis module (Carl Zeiss SpA).

Consecutive images, avoiding glomeruli, were recorded from the whole renal biopsy tissue at ×200 magnification. An optical threshold followed by filtering was applied to all images, and the staining for both CD45 and Vimentin and Periostin was calculated as percentage of the total scanned area.

Statistical Analyses:

Continuous variables were expressed as average values (mean±SD). Differences among groups were determined by Student's t test, Mann-Whitney test and ANOVA, where indicated. Differences among percentages were determined by χ² test and Fisher's exact test. Linear regression and logistic regression were employed in order to perform uni- and multi-varied analysis, respectively. All the data without a normal distribution were log transformed before they were introduced in uni- and multi-varied analyses.

In all statistical analyses significance was set for p values <0.05. Statistical analysis was performed using the software Statistica® version 10 and SAS 9.2 (SAS Institute Inc., Cary, N.C., USA).

Results

Cohort Characteristics:

In our study, we evaluated 149 renal transplanted patients (mean age 44±14 years old—M=83/F=66) undergone, exclusively for clinical reason, to transcutaneous Kidney Biopsy (KBx). In Table 1 the main characteristics of our cohort are summarized. They had a mean time of transplantation of 6±10 years, and were mostly transplanted by a deceased donor. The mean value of creatinine at T0 was 2.61±1.6 mg/dl, with an eGFR of 37±19 ml/min. Urinary protein excretion was 1.17±1.90 g/24 h. In 60% of them KBx was performed for a reduction of renal function whereas in 15% for recent increase of urinary protein excretion and in 14% for their association.

TABLE 1
General features of the studied population considered as a whole or divided
according to the graft survival (HD+ and HD−) − N = Mean ± SD (%)

| N | All pts n = 149 | HD− n = 117 | HD+ n = 32 | P |
|---|---|---|---|---|
| Number of patients | 149 | 177 | 32 | |
| Age at Bx (years) | 44 ± 14 | 50 ± 13 | 49 ± 12 | 0.71 |
| Age at the time of transplant (years) | 50 ± 12 | 44 ± 14 | 41 ± 11 | 0.29 |
| Gender (M/F) | 83/66 | 65/52 | 18/14 | 0.94 |
| Kind of transplant | 123/23 | 97/20 | 29/3 | 0.27 |
| (Deceased donor/living donor) | | | | |
| Time from transplant to Bx (years) | 6 ± 10 | 5 ± 6 | 7 ± 6 | 0.12 |
| CMV IgG pos N (%) | 107 (72) | 82 (70) | 25 (78) | 0.92 |
| HCV pos N (%) | 9 (6) | 4 (3) | 5 (15) | 0.01 |
| HbsAg pos N (%) | 4 (3) | 3 (2) | 1 (3) | 0.88 |
| Indication to Bx N (%): | | | | |
| Proteinuria | 22 (15) | 20 (17) | 2 (8) | 0.05 |
| Reduced RF | 89 (60) | 71 (61) | 18 (56) | |
| Both | 21 (14) | 12 (10) | 9 (28) | |
| Others | 17 (11) | 15 (12) | 2 (8) | |
| SBP (mmHg) | 133 ± 20 | 113 ± 16 | 136 ± 18 | 0.47 |
| DBP (mmHg) | 81 ± 11 | 81 ± 11 | 83 ± 10 | 0.52 |
| Creatinine 12 mo before Bx (mg/dL) | 1.65 ± 0.46 | 1.55 ± 0.39 | 1.92 ± 0.52 | 0.0004 |
| eGFR 12 mo before Bx (ml/min) | 51 ± 18 | 55 ± 18 | 43 ± 14 | 0.005 |
| Creatinine at Bx (mg/dL) | 2.61 ± 1.6 | 2.4 ± 1.5 | 3.5 ± 1.6 | 0.0004 |
| eGFR at Bx (ml/min) | 37 ± 19 | 41 ± 20 | 23 ± 8.7 | <0.0001 |
| eGFR 12 mo after re Bx (ml/min) | 41 ± 15 | 43 ± 15 | 26 ± 8 | 0.003 |
| Blood glucose (mg/dL) | 87 ± 23 | 89 ± 22 | 84 ± 26 | 0.32 |
| Mean UProt 12 mo before Bx | 0.58 ± 0.69 | 0.50 ± 0.65 | 0.80 ± 0.78 | 0.12 |
| UProt at Bx (g/24 h) | 1.17 ± 1.9 | 0.9 ± 1.08 | 2.29 ± 3.62 | 0.0006 |
| Mean UProt 12 mo after Bx | 0.79 ± 1.07 | 0.6 ± 0.67 | 2.5 ± 2.34 | <0.0001 |

CMV: Cytomegalovirus
HCV: Hepatitis virus C
HbsAg: Hepatitis B surface antigen
SBP: Systolic Blood Pressure
DBP: Diastolic Blood Pressure
eGFR: Glomerular Filtration Rate estimated with MDRD formula
uProt: urinary Protein excretion;

During the follow up time, 32 patients restarted dialysis (HD+). They were not different from the others in age and in time of transplantation. In HD+ group the indication to KBx was significantly more frequently the association of worsening of renal function and proteinuria. They had also higher levels of creatinine not only at T0 but also 12 and 6 months before KBx and of proteinuria at T0.

At the time of KBx (T0), the ongoing maintenance immunosuppressive therapy (IT), detailed in Table 2, was mainly based on: steroids, calcineurin inhibitors (CNI) and anti-proliferative drugs (MMF/MPA/AZA/mTOR-I).

TABLE 2
Main therapies in use in the studied patients at the time of Bx:

| Drug | N (%) |
|---|---|
| Steroid/CyA/Tac/MMF-MPA/mTor inhibitor/AZA | 130/65/75/105/13/6 |
| | (88/44/50/71/9/4) |
| Number of antihypertensive drugs (%): | |
| 0 | 11 (7) |
| 1 | 34 (23) |
| 2 | 57 (38) |
| 3 | 29 (19) |
| >3 | 18 (12) |
| RAS inhibitors (%) | 33 (23) |
| Diuretics (%) | 37 (25) |
| Beta-Blockers (%) | 106 (71) |
| Ca-Antagonists (%) | 62 (42) |
| Vit D therapy (%): Calcitriol/Calcifediol/Calcitriol | 44/14/2 (30/9/1) |
| and Calcifediol | |

Footnotes:
CyA: Cyclosporine
Tac: Tacrolimus
MMF: Mycophenolate mofetil Mofetil
MPA: Mycophenolic acid
AZA: Azathioprine

Histological Analysis:

General histological analysis was performed evaluating glomerulosclerosis, tubular atrophy, interstitial inflammation and fibrosis (Table 3). The percentage of glomerulosclerosis was 25±22. Moderate TA, IF and I-Inf was present in 22%, 26%, and 30% respectively, whereas severe lesions were found in 30%, 28% and 18% of patients. Fifty eight percent of KBx showed lesions compatible with chronic allograft injury, whereas 10% of patients were affected by a glomerulonephritis, 7% by cellular rejection, 4% humoral rejection. Only 5% of KBx resulted normal. Although no significant statistically difference was found between HD+ and HD− in histological diagnosis, HD+ showed more glomerulosclerosis (p=0.0009) and severe lesions both at tubular and interstitial levels than HD−.

TABLE 3
The degree of histological lesions in the two groups-
N = Mean ± SD (%):

| HISTOLOGICAL | All pts | | | |
|---|---|---|---|---|
| LESIONS | n = 149 | HD− n = 117 | HD+ n = 32 | P |
| Sclerotic | 25 ± 22 | 22 ± 20 | 37 ± 25 | 0.0009 |
| Glomeruli (%) | | | | |
| Tubular Atrophy | | | | |
| N (%) | | | | |
| No | 40 (27) | 34 (29) | 6 (19) | <0.0001 |
| Slight | 32 (21) | 30 (26) | 2 (6) | |
| Moderate | 33 (22) | 29 (25) | 4 (12) | |
| Severe | 44 (30) | 24 (20) | 20 (63) | |
| Interstitial | | | | |
| fibrosis N (%) | | | | |
| No | 32 (21) | 28 (24) | 4 (12) | 0.0004 |
| Slight | 37 (25) | 34 (30) | 3 (10) | |
| Moderate | 39 (26) | 32 (25) | 7 (22) | |
| Severe | 41 (28) | 23 (19) | 18 (56) | |
| Interstitial | | | | |
| infiltrate N (%) | | | | |
| No | 52 (34) | 41 (38) | 10 (31) | 0.06 |
| Slight | 27 (18) | 25 (22) | 2 (7) | |
| Moderate | 44 (30) | 34 (30) | 10 (31) | |
| Severe | 27 (18) | 10 (10) | 31 (31) | |

In all the cohort, glomerulosclerosis, at univariate regression was directly correlated with time of transplant (p<0.0001), age (p=0.02), PTH (p=0.01) and mean P during the year after KBx (p=0.015). An inverse correlation with eGFR evaluated both at 12 months before and after KBx (p=0.0003 and p=0.003 respectively) and at T0 (p=0.007) and with mean Ca during the year after KBx (p=0.0075) was found.

Patients that showed a pathologic grade of TA had significantly had a longer time of transplant (p=0.01), higher levels of Ca in the year before KBx (p=0.009) and higher percentage of glomerulosclerosis (p=0.0001). Interesting, no significant differences were found in renal function and urinary protein excretion.

Creatinine at T0 and 6 mths after KBx was higher in patients in which interstitial inflammation was present (p=0.01 and 0.001 respectively), as like as eGFR at T0 was lower (p=0.0003).

Time of transplantation was significantly longer in patients with interstitial fibrosis (p=0.01) that had also an higher percentage of glomerulosclerosis (p=0.001) and lower levels of 25OHVit D at T0 (p=0.02).

Specific Stainings:

Using univariate regression, a direct correlation each other between CD45, Vimentin and Periostin was found.

CD45 was correlated also with renal function (creatinine p=0.009—eGFR 0.005), PTH (p=0.0001 and Alkaline phosphatase (p=0.0002) at T0. Interestingly an inverse correlation with levels of 250H-Vit-D was found (p=0.004) (Table 4). Moreover, it was significantly higher in patient with any grade if interstitial infiltrate (p=0.005). No difference in tubular atrophy and interstitial fibrosis was found.

TABLE 4
CD45 correlations.

| | Parameter | | | | | | | | | |
|---|---|---|---|---|---|---|---|---|---|---|
| | −12 | | −6 | | T0 | | +6 | | +12 | |
| | r(X, Y) | p | r(X, Y) | p | r(X, Y) | p | r(X, Y) | p | r(X, Y) | p |
| Time of | | | | | −0.055 | 0.542 | | | | |
| transplantation | | | | | | | | | | |
| (years) | | | | | | | | | | |
| Age at biopsy | | | | | 0.032 | 0.723 | | | | |
| Créatinine | 0.114 | 0.311 | 0.009 | 0.932 | 0.198 | 0.025 | 0.221 | 0.027 | 0.029 | 0.802 |
| (mg/dl) | | | | | | | | | | |
| Mdrd (ml/min) | −0.074 | 0.540 | | | −0.263 | 0.006 | | | −0.097 | 0.412 |
| Mean Ca −12/6 | 0.028 | 0.803 | | | | | | | 0.011 | 0.915 |
| and +6/12 | | | | | | | | | | |
| (mg/dl) | | | | | | | | | | |
| Mean Ca −12/6 | −0.099 | 0.392 | | | | | | | −0.048 | 0.656 |
| and +6/12 | | | | | | | | | | |
| (mg/dl) | | | | | | | | | | |
| PTH (ng/dl) | | | | | 0.358 | 0.0001 | | | | |
| PCR (mg/dl) | | | | | 0.055 | 0.593 | | | | |
| Mean ProtU −12/6 | 0.088 | 0.502 | | | | | | | 0.015 | 0.887 |
| and +6/12 | | | | | | | | | | |
| (g/24 h) | | | | | | | | | | |
| 25-OH (ng/ml) | | | | | −0.255 | 0.014 | | | | |
| % sclerosis (% | | | | | 0.129 | 0.157 | | | | |
| of affected | | | | | | | | | | |
| glomeruli | | | | | | | | | | |
| Vimentin (of | | | | | 0.692 | 0.0001 | | | | |
| total positive | | | | | | | | | | |
| area) | | | | | | | | | | |
| Periostin (of | | | | | 0.464 | 0.0001 | | | | |
| total positive | | | | | | | | | | |
| area) | | | | | | | | | | |

Vimentin showed similar correlation than CD45. In fact it was correlated with creatinine and eGFR at baseline (both p=0.015), and with PTH and Alkaline Phosphatase at the same time (p=0.015 and p=0.04 respectively). Vimentin showed also a direct correlation with the percentage of glomerulosclerosis (p=0.01) and a tendency inversely with 250H-Vit-D (p=0.06) (Table 5). It was more expressed in patients with any grade of tubular atrophy (p=0.02) and interstitial infiltrate (p=0.01).

TABLE 5
Vimentin correlations.

| | Parameter | | | | | | | | | |
|---|---|---|---|---|---|---|---|---|---|---|
| | −12 | | −6 | | T0 | | +6 | | +12 | |
| | r(X, Y) | p | r(X, Y) | p | r(X, Y) | p | r(X, Y) | p | r(X, Y) | p |
| Time of | | | | | 0.104 | 0.242 | | | | |
| transplantation | | | | | | | | | | |
| (years) | | | | | | | | | | |
| Age at biopsy | | | | | 0.093 | 0.294 | | | | |
| Créatinine | 0.113 | 0.319 | −0.009 | 0.934 | 0.197 | 0.025 | 0.307 | 0.002 | 0.072 | 0.543 |
| (mg/dl) | | | | | | | | | | |
| Mdrd | −0.094 | 0.427 | | | −0.229 | 0.015 | | | −0.084 | 0.482 |
| (ml/min) | | | | | | | | | | |
| Mean Ca −12/6 | −0.099 | 0.383 | | | | | | | −0.067 | 0.522 |
| and +6/12 | | | | | | | | | | |
| (mg/dl) | | | | | | | | | | |
| Mean Ca −12/6 | −0.012 | 0.920 | | | | | | | 0.006 | 0.955 |
| and +6/12 | | | | | | | | | | |
| (mg/dl) | | | | | | | | | | |
| PTH (ng/dl) | | | | | 0.234 | 0.014 | | | | |
| PCR (mg/dl) | | | | | 0.013 | 0.90 | | | | |
| Mean ProtU −12/6 | 0.066 | 0.611 | | | | | | | 0.028 | 0.792 |
| and +6/12 | | | | | | | | | | |
| (g/24 h) | | | | | | | | | | |
| 25-OH | | | | | −0.191 | 0.063 | | | | |
| (ng/ml) | | | | | | | | | | |
| % sclerosis (% | | | | | 0.147 | 0.103 | | | | |
| of affected | | | | | | | | | | |
| glomeruli | | | | | | | | | | |
| CD45 (of total | | | | | 0.692 | 0.0001 | | | | |
| positive area) | | | | | | | | | | |
| Periostin (of | | | | | 0.414 | 0.0006 | | | | |
| total positive | | | | | | | | | | |
| area) | | | | | | | | | | |

Periostin, in addition to correlate directly with CD45 and Vimentin (p<0.0001 and 0.0006 respectively), was directly correlated with the percentage of glomerulosclerosis (p=0.02) and inversely with 250H-Vit-D (p=0.05). Relations neither with renal function and urinary protein excretion nor with general histologic anomalies were found (Table 6).

TABLE 6
Periostin correlations.

| | Parameter | | | | | | | | | |
|---|---|---|---|---|---|---|---|---|---|---|
| | −12 | | −6 | | T0 | | +6 | | +12 | |
| | r(X, Y) | p | r(X, Y) | p | r(X, Y) | p | r(X, Y) | p | r(X, Y) | p |
| Time of | | | | | 0.091 | 0.454 | | | | |
| transplantation | | | | | | | | | | |
| (years) | | | | | | | | | | |
| Age at biopsy | | | | | −0.079 | 0.517 | | | | |
| Créatinine | 0.181 | 0.234 | 0.122 | 0.409 | 0.115 | 0.339 | 0.0039 | 0.011 | 0.001 | 0.994 |
| (mg/dl) | | | | | | | | | | |
| Mdrd | −0.141 | 0.392 | | | | | | | −0.155 | 0.294 |
| (ml/min) | | | | | | | | | | |
| Mean Ca −12/6 | −0.024 | 0.881 | | | | | | | 0.154 | 0.297 |
| and +6/12 | | | | | | | | | | |
| (mg/dl) | | | | | | | | | | |
| Mean Ca −12/6 | 0.082 | 0.618 | | | | | | | | |
| and +6/12 | | | | | | | | | | |
| (mg/dl) | | | | | | | | | | |
| PTH (ng/dl) | | | | | 0.238 | 0.083 | | | | |
| PCR (mg/dl) | | | | | 0.016 | 0.907 | | | | |
| Mean ProtU −12/6 | 0.124 | 0.522 | | | | | | | 0.116 | 0.431 |
| and +6/12 | | | | | | | | | | |
| (g/24 h) | | | | | | | | | | |
| 25-OH | | | | | −0.229 | 0.114 | | | | |
| (ng/ml) | | | | | | | | | | |
| % sclerosis (% | | | | | 0.207 | 0.093 | | | | |
| of affected | | | | | | | | | | |
| glomeruli | | | | | | | | | | |
| CD45 (of total | | | | | 0.464 | 0.0001 | | | | |
| positive area) | | | | | | | | | | |
| Vimentin (of | | | | | 0.414 | 0.0006 | | | | |
| total positive | | | | | | | | | | |
| area) | | | | | | | | | | |

In patients that during the follow up-time had lost their graft, all the three markers were significantly more positive in the tissue at the moment of biopsy. Using logistic regression, this relation resulted independent form creatinine values at T0 only for CD45 (p=0.003) and Periostin (p=0.006).

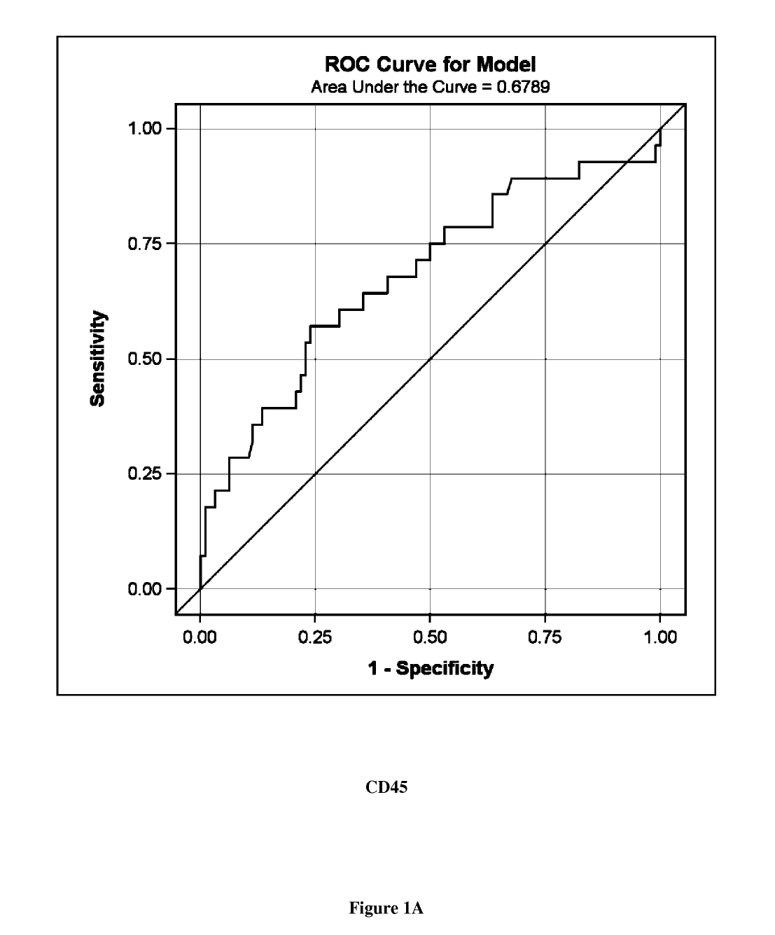

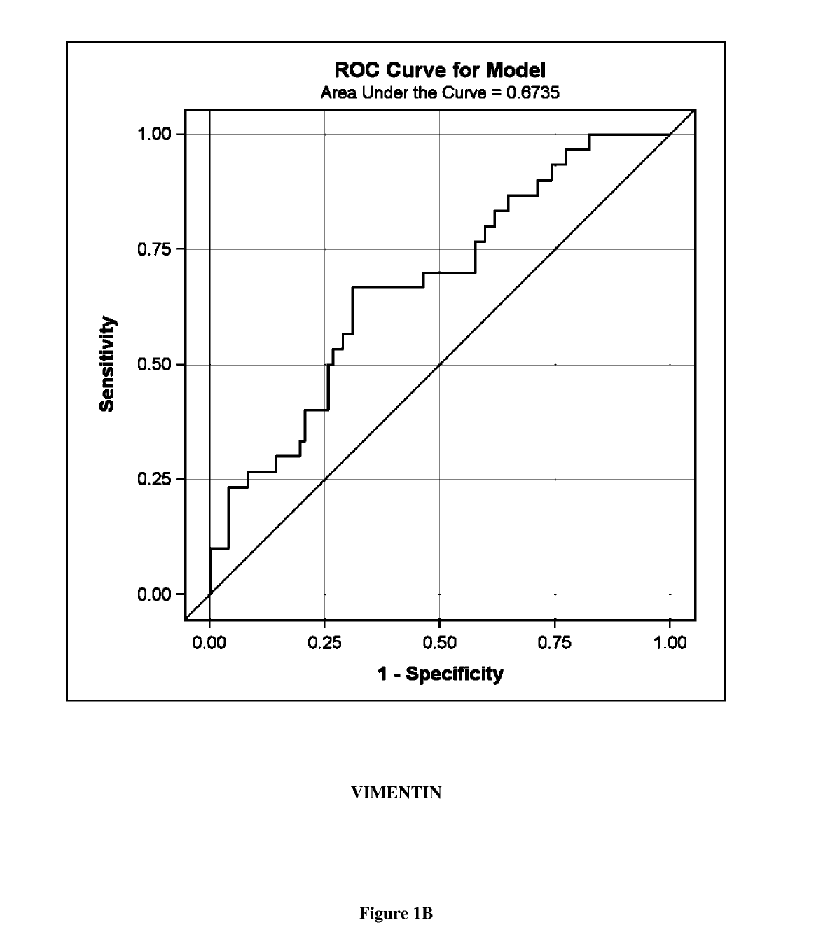

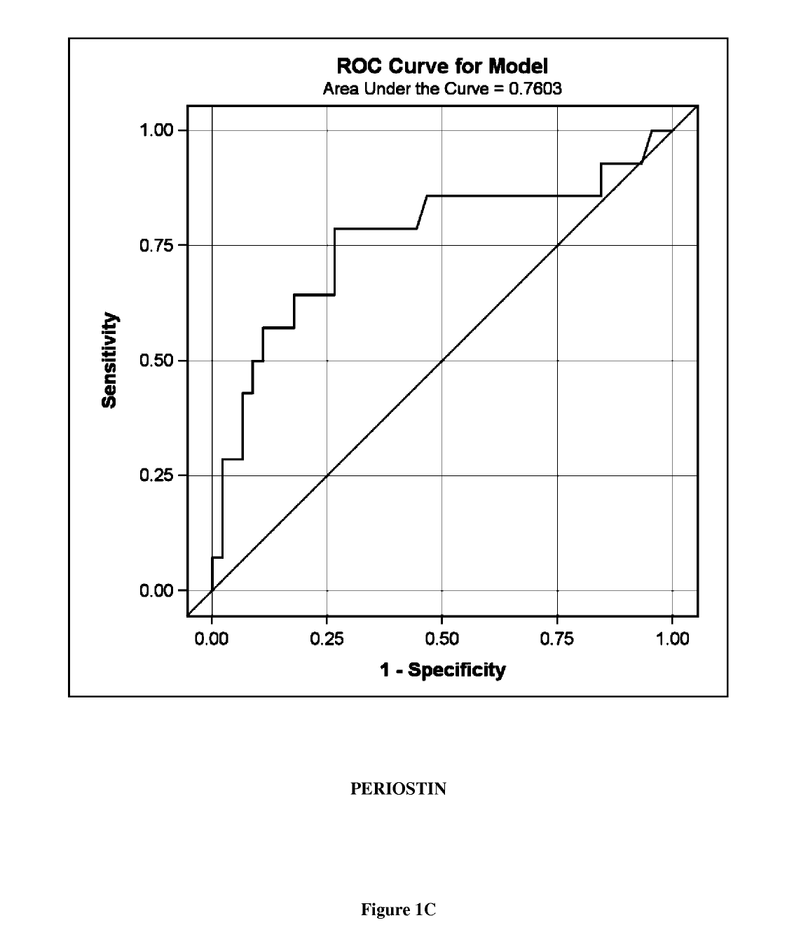

To evaluate specificity and sensitivity in predicting graft outcome, ROC curves were performed (FIG. 1). They demonstrated the good predictive role of all these three markers (CD45 and Vimentin: AUC=0.67) and especially of Periostin (AUC=0.76).

### REFERENCES

Throughout this application, various references describe the state of the art to which this invention pertains. The disclosures of these references are hereby incorporated by reference into the present disclosure.
1. Levey A S, Bosch J P., Lewis J B, Greene T, Rogers N, Roth D. A more accurate method to estimate glomerular filtration rate from serum creatinine: a new prediction equation. Modification of Diet in Renal Disease Study Group. Ann Intern Med 1999; 130 (6): 461-70.
2. Vaidya S, Partlow D, Susskind B, Noor M, Barnes T, Gugliuzza K. Prediction of crossmatch outcome of highly sensitized patients by single and/or multiple antigen bead luminex assay. Transplantation 2006; 82(11): 1524-1528
3. Gibney E M, Cagle L R, Freed B, Warnell S E, Chan L, Wiseman A C. Detection of donor-specific antibodies using HLA-coated microspheres: another tool for kidney transplant risk stratification. Nephrol Dial Transplant 2006; 21(9):2625-2629
4. Walker P D, Cavallo T, Bonsib S M; Ad Hoc Committee on Renal Biopsy Guidelines of the Renal Pathology Society. Practice guidelines for the renal biopsy. Mod Pathol. 2004; 17(12):1555-63
5. Sis B, Mengel M, Haas M et al. Banff '09 meeting report: antibody mediated graft deterioration and implementation of Banff working groups. Am J Transplant. 2010; 10(3):464-71
6. Solez K, Colvin R B, Racusen L C et al. Banff 05 Meeting Report: differential diagnosis of chronic allograft injury and elimination of chronic allograft nephropathy (CAN). Am J Transplant 2007; 7 (3): 518-26

1. A method for determining whether a renal transplant patient is at risk of transplant rejection, comprising a step of determining an expression level of a POSTN gene in a renal transplant biopsy obtained from said renal transplant patient, wherein an increase in the expression level of the POSTN gene compared to the predetermined reference value indicates that the renal transplant patient is at risk of transplant rejection.

2. (canceled)

3. The method according to claim 1, wherein the expression level of the POSTN gene is determined by measuring an amount of POSTN by immunohistochemistry.

4. The method according to claim 1, wherein the transplant rejection is an antibody-mediated rejection (ABMR) or a T cell-mediated rejection (TCMR).

5. The method according to claim 1, wherein the transplant rejection is an acute transplant rejection or a chronic transplant rejection.

6. The method according to claim 1, further comprising a step of determining the expression level of a CD45 gene and/or a Vimentin gene in a renal transplant biopsy obtained from said transplanted patient.

7-8. (canceled)

9. The kit according to claim 11, wherein said means for determining the expression level of the POSTN gene is an anti-POSTN antibody.

10. (canceled)

11. A kit comprising means for determining an expression level of a POSTN gene and means for determining an expression level of a CD45 gene and/or a Vimentin gene.

12. A method for adjusting an immunosuppressive treatment administered to a renal transplant recipient after transplantation, comprising:
(i) determining whether the renal transplant recipient is at risk of transplant rejection by performing the method of claim 1
and
(ii) adjusting the immunosuppressive treatment of the renal transplant recipient according to results obtained in said determining step, wherein if POSTN is present in the renal transplant biopsy, then the renal transplant recipient is treated with an effective amount of an immunosuppressive treatment, or if POSTN is absent from the renal transplant biopsy, then the renal transplant recipient is not treated with an immunosuppressive treatment.

13. The method of claim 12, wherein the immunosuppressive treatment comprises administering a compound selected from the group consisting of monoclonal anti-CD20 antibodies, anti-thymocyte globulin (ATG), proteasome inhibitors, anti-05 antibodies, monoclonal anti-CD3 antibodies, glucocorticoids, cytostatics, calcineurin inhibitors (CNI) and mTOR inhibitors.

14. A method for preventing renal transplant rejection in a renal transplant patient, comprising the steps of:
(i) performing the method of claim 1 to determine whether the renal transplant patient is at risk of renal transplant rejection,
(ii) comparing the expression level of the POSTN gene to a predetermined reference value, and, if an increase in the expression level of the POSTN gene compared to the predetermined reference value is detected, then
(iii) administering to said patient an amount of an immunosuppressive treatment sufficient to prevent renal transplant rejection.

15. The method of claim 14, wherein the immunosuppressive treatment comprises administering to the renal transplant patient a compound selected from the group consisting of monoclonal anti-CD20 antibodies, anti-thymocyte globulin (ATG), proteasome inhibitors, anti-C5 antibodies, monoclonal anti-CD3 antibodies, glucocorticoids, cytostatics, calcineurin inhibitors (CNI) and mTOR inhibitors.

16. An analytical method comprising
obtaining a renal transplant biopsy from a renal transplant patient, and
determining an expression level of a POSTN gene in the renal transplant biopsy.
